(19) United States
(12) Patent Application Publication
(10) Pub. No.: US 2014/0314890 A1
(43) Pub. Date: Oct. 23, 2014

(54) COMBINATION ANTI-CANCER THERAPY

(71) Applicants: THE FLORIDA INTERNATIONAL UNIVERSITY BOARD OF TRUSTEES, Miami, FL (US); NOVA SOUTHEASTERN UNIVERSITY, Fort Lauderdale, FL (US)

(72) Inventors: Miroslav Gantar, Miami, FL (US); Appu Rathinavelu, Weston, FL (US); Sivanesan Dhandayuthapani, Plantation, FL (US)

(73) Assignees: THE FLORIDA INTERNATIONAL UNIVERSITY BOARD OF TRUSTEES, Miami, FL (US); NOVA SOUTHEASTERN UNIVERSITY, Fort Lauderdale, FL (US)

(21) Appl. No.: 14/354,939

(22) PCT Filed: Oct. 30, 2012
(86) PCT No.: PCT/US2012/062567

§ 371 Date: Apr. 29, 2014

Related U.S. Application Data
(60) Provisional application No. 61/553,566, filed on Oct. 31, 2011.

(51) Int. Cl. A61K 35/74; A61K 45/06; A61K 38/16

(52) U.S. Cl. CPC A61K 35/748; A61K 38/164; A61K 45/06
USPC 424/780; 530/350

(57) ABSTRACT

Discloses herein is a combination therapy for treating cancer by administration of a phycocyanin and a chemotherapeutic.

### CROSS-REFERENCE TO RELATED APPLICATIONS

The benefit under 35 U.S.C. §119 is claimed to U.S. provisional application No. 61/553,566, filed Oct. 31, 2011, the disclosure of which is incorporated by reference in its entirety.

### BACKGROUND

Chemotherapy represents currently the most often used modality in cancer treatment. Most of the currently used chemotherapeutic drugs have their origin from plants, microbes or marine organisms. However, the number of antineoplastic agents currently available to oncologists is quite limited and there is a great need to identify either new antineoplastic drugs or some agents, preferentially from food or food supplements that will enhance and improve the efficacy of currently available drugs. In that respect, cyanobacteria have been screened for, and many identified as a potential novel source of anticancer drugs (Patterson et al., 1991; Patterson et al., 1994). In 1980s, the cyanobacterium (or blue-green alga) Spirulina started to be marketed as a health food with numerous claims about the beneficial effects on human health, among others as a potential source of novel anticancer drug. Many of these medicinal properties of Spirulina are now associated with one particular compound—phycocyanin.

Phycocyanin is a blue colored photosynthetic accessory pigment that absorbs light at 620 nm and emits fluorescence at about 560 nm. It is a protein, molecular weight of which depends on the organism from which it was isolated and ranges between 110-230 kDa, containing two subunits. Subunit α carries one and subunit β has two tetrapyrol chromophores (Glazer and Fang, 1973).

One of the first reports on beneficial effect of phycocyanin (Belay et al., 1993) cite the Japanese patent #58-65216 (Dainippon Ink & Chemicals, 1983), according to which this blue pigment from Spirulina significantly increased the survival rates of mice that had been injected with liver tumor cells. It has been suggested that stimulation of the immune system by phycocyanin was a mechanism that inhibited the growth of tumor cells. Liu et al. (2000) also reported on antineoplastic activity of phycocyanin isolated from Spirulina. They found that this pigment significantly inhibited the growth of human leukemia K562 cells through mechanisms other than apoptosis. However, induced apoptosis was described as a mechanism of phycocyanin action by a number of authors. Reddy et al. (2003) used mouse macrophage cells, which were treated with LPS to express high levels of COX-2, over expression of which is known to be responsible for down regulation of apoptosis. When these cells were exposed to phycocyanin, it induced apoptosis in a dose dependent manner. A different mechanism of apoptosis induction was described in AK5 tumor cells. Bobbili et al. (2003) explained the C-PC induction of apoptosis by down-regulating of Bcl-2 (a known inhibitor of apoptosis) and by generation of radical oxygen species (ROS). This finding was also confirmed with the human chronic myeloid leukemia cell line-K562 (Subhashini et al., 2004). At present, it is not known whether the whole phycocyanin molecule is required for the anticancer activity. Nevertheless, it was shown that the -subunit of phycocyanin that was cloned in E.coli, had the same or even more potent power of apoptotic induction when tested on four different cancer cell lines (Wang et al., 2007). Cells of the treated cancer cell lines underwent apoptosis with an increase in caspase-3 and caspase-8 activities with an arrest at the G0/G1 phase. Caspase-dependant apoptosis was also confirmed in phycocyanin treated HeLa cells (Li et al. 2006).

### BRIEF DESCRIPTION OF THE FIGURES

FIG. 1 shows percentage of live cells after exposure to various treatments, as noted.

FIG. 2 shows levels of ROS in LNCaP cells after exposure to various treatments, as noted.

FIG. 3 shows DNA fragmentation in LNCaP cells after exposure to various treatments, where lane 1 is DNA ladder, lane 2 is control, lane 3 is 1 μM TPT, lane 4 is 10 μM TPT, lane 5 is 250 μg/mL PC, lane 6 is 500 μg/mL PC, lane 7 is 250 μg/mL PC+1 μM TPT, and lane 8 is 500 μg/mL PC+1 μM TPT.

FIG. 4 shows activities of Caspase-3 and Caspase-9 in PC and TCT treated LNCaP cells.

### DETAILED DESCRIPTION

Induction of apoptosis is a desired consequence when cancer is treated using chemotherapeutic agents. In recent years several natural products with the ability to induce apoptosis in cancer cells have been tested for anti-cancer effects. Phycocyanin that is isolated from the cyanobacterium Limnothrix sp. 37-2-1 seemed to exhibit significant anti-cancer properties in our in vitro systems. Therefore, we conducted several experiments to test whether phycocyanin combination with lower than usual doses of topotecan (1 μM) can offer the same level of cytotoxic effects as high dose topotecan (10 μM). For this purpose cytotoxicities of phycocyanin and topotecan were tested using the LNCaP (prostate cancer), cell lines using 16 h drug exposure. Furthermore, the level of reactive oxygen species (ROS) generation and the activities of the caspase-9 and caspase-3 were also measured after phycocyanin and topotecan treatments. Both topotecan and phycocyanin were able to induce the generation of ROS by themselves and also in combination with each other. Once, the ROS levels were increased, we were also able to detect the increase in the activities of caspase-9 and caspase-3. Taken together, our findings suggest that, phycocyanin from Limnothrix sp. in combination with lower dose of topotecan can induce apoptosis and cancer cell death through generation of ROS and activation of the major apoptotic enzymes such as caspase-9 and caspase-3 and finally causing DNA fragmentation.

In this work, we used C-PC from our novel cyanobacterial isolate Limnothrix sp. 37-2-1 to test its anticancer properties. We have shown that Limnothrix can be a better source for C-PC than traditionally used Spirulina (Gantar et al. submitted). In our preliminary work, we have confirmed that C-PC from Limnothrix indeed had antiproliferative activity against prostate cancer cell line (LNCaP). However, the required concentration of phycocyanin for anticancer activity was well above the range of anticancer drugs normally used. Therefore, we hypothesized that C-PC could be potentially used as an enhancer of the existing anticancer drugs. Here we are presenting data of in vitro experiments, which confirm our hypothesis.

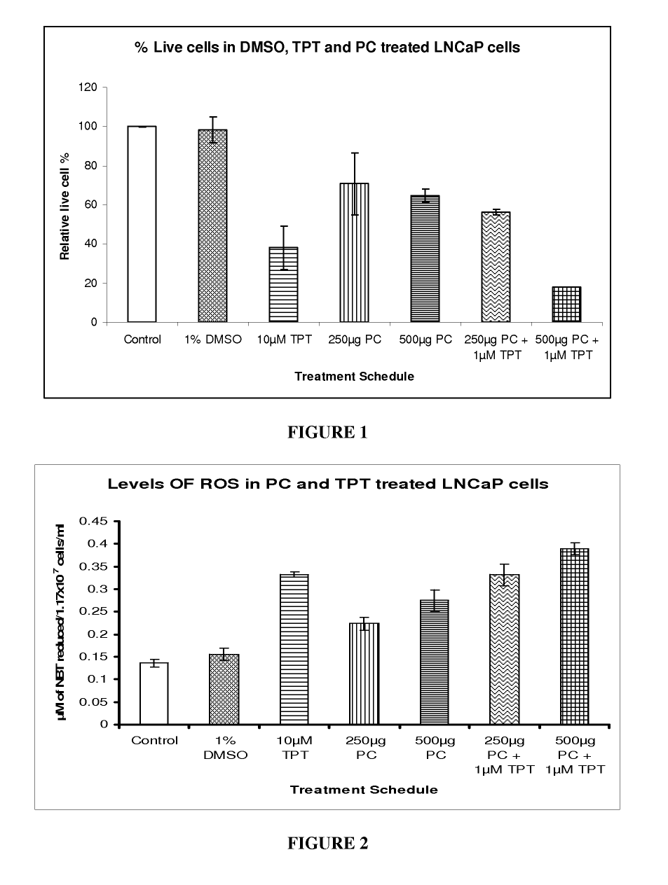

Treatment of LNCaP cells with phycocyanin and topotecan resulted in a significant inhibition of cell growth (FIG. 1). This effect was more pronounced at post-treatment with C-PC plus topotecan as in combination. Further the generation of reactive oxygen species (ROS) upon treatment with phycocyanin and topotecan alone and as in combination was also determined using the NBT reduction colorimetric assay. Treatment of LNCaP cells with phycocyanin and topotecan showed a significant increase in the rate of ROS generation (FIG. 2).

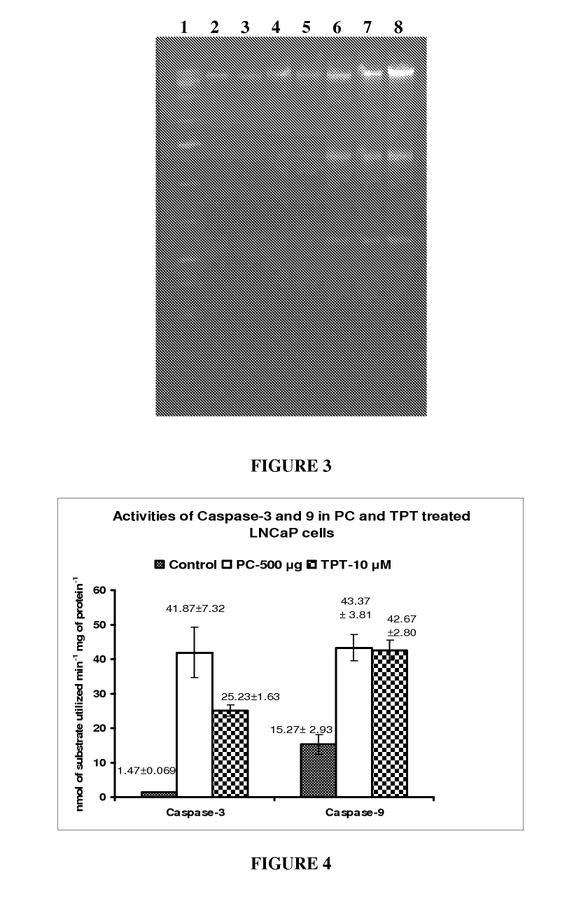

The inhibition of cell viability could result from the induction of apoptosis. Therefore, the cells were treated with the phycocyanin and topotecan and the mechanism of cell growth inhibition was treated by the apoptotic feature in the cells using DNA fragmentation analysis. Internucleosomal DNA fragmentation was clearly visible (FIG. 3) after treatment with different concentrations of phycocyanin in combination with topotecan.

To identify whether caspases were involved in phycocyanin induced apoptosis, the control, phycocyanin and topotecan treated cell lysates were analyzed to measure the caspase-3 and caspase-9 activities using colorimetric assay. As expected, caspase-3 and caspase-9(FIG. 4) activities in phycocyanin and topotecan treated cells were found increased when compared to untreated control cells. Our data suggests, that phycocyanin-induced apoptosis is characterized by the caspase-dependent pathway.

Chemoprevention, the use of drugs or natural substances to inhibit carcinogenesis, is an important and rapidly growing subject of cancer research. There has recently been a surge of interest in marine bioresources, particularly seaweeds, as sources of bioactive substances. C-PC is one of the major water-soluble biliprotein present in Limnothrix sp. 37-2-1. In an effort to gain insight into effects of C-PC and to understand the mechanism underlying phycocyanin induced apoptosis and synergistic enhancement of the anti-cancer effect of topotecan, in the present study, we have demonstrated that the combination between the standard chemotherapeutic agent topotecan and phycocyanin synergistically enhanced the cancer cell death. Specifically, when only 10% of typical dose of topotecan was combined with 250 or 500 ug of phycocyanin (FIG. 1), the cancer cells were killed at much higher rate than when topotecan and phycocyanin were used separately at the same dose. This is the first evidence that phycocyanin can enhance the cytotoxic effect of the known anticancer drug. Though only combination with topotecan has been tested similar combination can be achieved with of Topotecan, Irinotecan, Taxol, Vincristin, Vinblastine, Etoposide, Doxorubicin, Danaurubicin, Idarubicin, Mitomycin, Mitoxantrone, Cisplain, Carboplatin, Procarbazine, Carmustine, Lomustine, DTIC, Cyclophosphamide, Ifosphamide etc.

Accumulative evidences suggest that defects in the process of apoptosis may be closely associated with carcinogenesis and that many cancer cells have defective machinery for self-destruction (Yano et al., 1994). Apoptosis is a specific mode of cell death recognized by a characteristic pattern of morphological, biochemical, and molecular changes. Most of the existing cancer drugs target apoptosis. Our findings that phycocyanin affects the LNCaP cells by inducing apoptosis was confirmed by DNA fragmentation (FIG. 3) shown both when phycocyanin and topotecan were used individually and in combination. Therefore, among various other possibilities one of the probable cause of cell death during phycocyanin and anticancer drug combination treatment is induction of ROS production leading to caspase 3 activation that results in DNA fragmentation. It appears that phycocyanin is potentiating the effect of apoptosis inducing anticancer drugs in every step of the way.

Our study of the mechanisms by which phycocyanin affects cancer cells, confirms earlier reports (Bobbili et al., 2003) that this is a ROS-dependent apoptosis. The highest level of ROS production was observed when the LNCaP cells were treated with the combination of topotecan and phycocyanin. Furthermore, phycocyanin treatment increased theactivities of both caspase-3 and caspase-9. Since the caspases are the most important proteases found in apoptotic cells (Yano et al., 1994) their increase during phycocyanin treatment is another proof of potentiating apoptotic cell death.

In conclusion, our results suggest that administration of C-PC together with chemotherapeutic agent such as topotecan can reduce the effective dose necessary for the cancer cells to move toward apoptosis and mitigate the cytotoxic effect on LNCaP (prostate) cell. Further in vivo and in vitro molecular studies will be needed to identify the appropriate application conditions and doses of C-PC in the treatment process.

Thus, provided herein are combination therapy for treating cancer by administering both a chemotherapeutic and a cyanobacteria extract. The cancer can be, e.g., a carcinoma, adenocarcinoma, or sarcoma. Some specifically contemplated cancers include prostate cancer, breast cancer, ovarian cancer, colorectal cancer, lung cancer, liver cancer, pancreatic cancer, esophageal cancer, or skin cancer.

More particularly, provided herein are methods of treating cancer comprising administering a therapeutically effective amount of a chemotherapeutic and a therapeutically effective amount of a cyanobacteria extract to a subject thereof. In some cases, the amount of chemotherapeutic administered to the subject in the disclosed combination therapy methods is less than the amount of chemotherapeutic administered to the subject alone (e.g., not in a combination therapy). The amount of chemotherapeutic administered in the disclosed combination therapy methods can be 10%, 15%, 20%, 25%, 30%, 35%, 40%, 45%, 50%, 55%, 60%, 65%, 70%, 75%, or 80% of an amount of the chemotherapeutic administered alone (e.g., in the absence of the cyanobacteria extract).

The chemotherapeutic administered in any of the methods disclosed herein can induce apoptosis and/or induce DNA fragmentation. Specifically contemplated chemotherapeutics for the methods disclosed herein include topotecan, irinotecan, taxol, vincristin, vinblastine, etoposide, doxorubicin, danaurubicin, idarubicin, mitomycin, mitoxantrone, cisplain, cisplatin, carboplatin, procarbazine, carmustine, lomustine, DTIC, cyclophosphamide, and ifosphamide.

The cyanobacteria extract can be one or more of phycocyanin, allophycocyanin, and phycoerthrin. In some cases, the cyanobacteria extract comprises phycocyanin.

The chemotherapeutic and cyanobacteria extract can be administered in any order. In some cases, they are administered sequentially (chemotherapeutic then cyanobacteria extract or cyanobacteria extract then chemotherapeutic) or simultaneously (and in some cases can be co-formulated).

In cases where the two agents are administered sequentially, the time between administration of the first agent and the second agent can be 15 minutes, 20 minutes, 30 minutes, 45 minutes, 1 hour, 1.5 hours, 2 hours, 2.5 hours, 3 hours, 4 hours, 6 hours, 8 hours, 10 hours, 12 hours, 14 hours, 16 hours, 24 hours, 1.5 days, 2 days, 3 days, 4 days, 5 days, 6 days, 7 days, 1.5 weeks, 2 weeks, 3 weeks, or 1 month. In some cases, the time between administration of the two agents is 24 hours or less.

As herein, the compounds described herein may be formulated in pharmaceutical compositions with a pharmaceutically acceptable excipient, carrier, or diluent. The compound or composition comprising the compound is administered by any route that permits treatment of the disease or condition. One route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, topically, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or a compound as disclosed herein can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

In an embodiment, the pharmaceutical compositions are formulated with one or more pharmaceutically acceptable excipient, such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of bacterial infection (e.g., anti-bacterial or anti-microbial agents).

Formulations, e.g., for parenteral or oral administration, are most typically solids, liquid solutions, emulsions or suspensions, while inhalable formulations for pulmonary administration are generally liquids or powders. A pharmaceutical composition can also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The pharmaceutical compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In some embodiments, the compounds described herein may be formulated for oral administration in a lipid-based formulation suitable for low solubility compounds. Lipid-based formulations can generally enhance the oral bioavailability of such compounds.

As such, pharmaceutical compositions comprise a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

In some embodiments, cyclodextrins may be added as aqueous solubility enhancers. Exemplary cyclodextrins include hydroxypropyl, hydroxyethyl, glucosyl, maltosyl and maltotriosyl derivatives of α-, β-, and γ-cyclodextrin. A specific cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the invention in the composition.

### EXAMPLES

### Phycocyanin Isolation and Purification:

Phycocyanin was isolated from the novel strain of cyanobacterium Limnothirix sp. 37-2-1 by a procedure described elsewhere (Gantar et al. 2011—to be submitted). In short, the cyanobacterium was grown in 30-liter photobioreactor, biomass was harvested and extracted. The purification of the pigment was performed by fractional precipitation of the cell crude extract by ammonium sulfate, treatment with activated carbon and chitosan. Desalting and concentrating of the pigment was performed by tangential flow filtration. The purity of phycocyanin was checked by SDS-PAGE and by calculating the ration of the absorbances at 620 and 280 nm. Only those preparations of phycocyanin that had the 620/280 ratio greater than 4.0, were used for further experiments. The purified phycocyanin was freeze-dried and kept in a refrigerator until use.

### Cell Line and Reagents

The prostate cancer (LNCaP) cell line was a generous gift from Dr. Thomas Powell (Cleveland Clinic Foundation, Cleveland, Ohio). The RPMI-1640 growth medium, Amphotericin-B, L-glutamine, antibiotic-antimycotic solution containing penicillin and streptomycin were obtained from Atlanta Biologicals (Lawrenceville, Ga.). The fetal bovine serum was purchased from Hyclone (Logan, Utah) and bromelain was purchased from Sigma Chemical Co. (St. Louis, Mo.). The substrates and inhibitors of caspase-9 and caspase-3 were purchased from Enzo Life Sciences AG (Lausen, Switzerland). The Qiagen DNeasy kit was used for the extraction of the DNA to determine the DNA fragmentation. All other chemicals used in our research were of research grade purchased through standard suppliers.

### Cell Culture and Treatment

The LNCaP cells were grown as a complete monolayer in RPMI-1640 growth medium supplemented with 10% fetal bovine serum, 10,000 U/mL of penicillin, 10,000 μg/mL of streptomycin, 1% (+)-L-glutamine, and 1% amphotericin-B. The cells were grown at 37° C. under a humidified air/CO2 (19:1) atmosphere. All experiments were conducted using cells in logarithmic phase. In each well of a six-well plate, approximately 1×106 cells per well were grown for 24 hours.

### Determination of Cell Death and ROS

To determine the cell viability, monolayer culture of LNCaP cells were treated with different concentrations of the C-PC (250 and 500 μg mL−1) and topotecan (1 μM and 10 μM) individually and in combination of C-PC (250 and 500 μg mL−1) with topotecan at 1 μM for 16 hours. After the incubation period, cell death was determined by using trypan blue dye exclusion test. To determine the levels of ROS generation, the cells cultured in 6-well plate were incubated with different concentrations of C-PC and topotecan as described above for 16 hours. At the end of incubation period, levels of ROS generated were measured using the NBT reduction assay.

### Assay of Caspase-9 and Caspase-3 Activities

LNCaP cells were treated with different concentrations of the C-PC (250 μg mL−1) and topotecan (10 μM) for 16 hours. After the incubation time, cells were harvested, washed and resuspended in cell lysis buffer and kept on ice for 10 minutes. The cell lysate was centrifuged at 5000 rpm for 5 minutes, the supernatant was assayed for protein concentration and further diluted with the lysis buffer to adjust the protein concentration. Equal amounts of protein from each sample was added to 96-well plates and mixed with the 2×reaction buffer [100 mM HEPES (pH 7.4), 200 mM NaCl, 20 mM dithiotheritol, 2 mM EDTA, and 0.2% chaps] and 2 mM of the respective substrates acetyl-Leu-Glu-His-Asp-p-nitroaniline (Ac-LEHD-pNA) and acetyl-Asp-Glu-Val-Asp-p-nitroaniline (Ac-DEVD-pNA). For measuring the specific activity of caspase-9 and caspase-3, the respective inhibitors Ac-LEHD-CHO and Ac-DEVD-CHO were used. Release of the cleaved p-nitroanilide from the tetrapeptide substrates was measured using the 96-well microplate reader at 405 nm.

### DNA Fragmentation Assay

A monolayer of LNCaP cells were grown in T-25 culture flasks and incubated with different concentrations of phycocyanin and topotecan individually and in combination of C-PC and topotecan. After incubation the cells were harvested and washed with PBS. Cells were resuspended in 200 μL of PBS and 20 μL of proteinase K was added. The DNA was extracted using the Qiagen DNeasy Kit, following the manufacturer's protocol. The samples were subjected to electrophoresis at 80 V for 2 hours in 1.5% agarose gel containing 5 μL of ethidium bromide. Separated DNA fragments were viewed with an UVP image analyzer.

### REFERENCES

Bobbili V V P, Mubarak Ali A, Leela Kumari A, Pallu Reddanna and Ashok Khar, Phycocyanin-mediated apoptosis in AK-5 tumor cells involves down-regulation of Bcl-2 and generation of ROS, Molecular Cancer Therapeutics, 2003 2: 1165-1170.

Belay A, Ota Y, Miyakawa K and Shimamatsu H, Current knowledge on potential health benefits of Spirulina. J Appl Phycol 1993; 5: 235-241.

Dainippon Ink & Chemicals, 1983

Glazer A N and Fang S, Formation of hybrid proteins from the α and β subunits phycocyanin of unicellular and filamentous blue-green algae, The J Biol. Chem., 1973, 248: 663-671. Li B, Gao M H, Zhang X C and Chu X M, Molecular immune mechanism of C-phycocyanin from Spirulina platensis induced apoptosis in HeLa cells in vitro, Biotechnol Appl Biochem. 2006; 43: 155-164.

Liu Y, Xu L, Cheng N, Lin L, Zhang C. Inhibitory effect of phycocyanin from Spirulina platensis on the growth of human leukemia K562 cells. J. Appl. Phycol. 2000, 12: 125-130.

Patterson G M L, Baldwin C L, Bolis C M, Caplan F R, Karuso H, Larsen L K, Levine I A, Moore R E, Nelson C S, Tschappat K D, Tuang G D, Furusawa E, Furusawa S, Norton T R and Raybourne R B, Antineoplastic activity of cultures blue-green algae (Cyanophyta), J Appl Phycol 1991, 27: 530-536.

Patterson G. M. L., Larsen L. K., Moore R. E. Bioactive natural products from blue-green algae. J Phycol, 1994, 6: 151-157.

Reddy M C, Subhashini J, Mahipal S V K, Bhat V B, Reddy P S, Kiranmai G, Madyastha K M, Reddanna P, C-Phycocyanin, a selective cyclooxygenase-2 inhibitor, induces apoptosis in lipopolysaccharide-stimulated RAW 264.7 macrophages, Biochem Biophys Res Commun, 2003, 304: 385-392

Subhashini J, Mahipal S V K, Reddy M C, Reddy M M, Rachamallu A and Reddanna P, Molecular mechanisms in C-phycocyanin induced apoptosis in human chronic myeloid leukemia cell line-K562, Biochem Pharmacol, 2004, 68: 453-462.

Wang H, Liu Y, Gao X, Carter C L and Liu Z-R, The recombinant β subunit of C-phycocyanin inhibits cell proliferation and induces apoptosis, Cancer Letters, 2007, 18: 150-158.

Yano H, Mizoguchi A, Fukuda K, Haramaki M, Ogasawara S, Momosaki S, et al., The herbal medicine sho-saiko-to inhibits proliferation of cancer cell lines by inducing apoptosis and arrest at the G0/G1 phase. Cancer Res 1994; 54: 448-454.

### Embodiments

1. A method of treating cancer in a subject comprising administering (a) a therapeutically effective amount of a chemotherapeutic and (b) a therapeutically effective amount of a cyanobacteria extract to the subject.

2. The method of paragraph 1, wherein the therapeutically effective amount of the chemotherapeutic is an amount less than a therapeutically effective amount of the chemotherapeutic when administered in the absence of the cyanobacteria extract.

3. The method of paragraph 2, wherein the amount of the chemotherapeutic administered is 50% or less the amount administered to the subject not administered the cyanobacteria extract.

4. The method of paragraph 3, wherein the amount of chemotherapeutic administered is 25% or less.

5. The method of any one of paragraphs 1 to 4, wherein the chemotherapeutic induces apoptosis.

6. The method of any one of paragraphs 1 to 5, where the chemotherapeutic induces cytotoxicity.

7. The method of any one of paragraphs 1 to 6, wherein the chemotherapeutic induces DNA fragmentation.

8. The method of any one of paragraphs 1 to 7, wherein the chemotherapeutic is one or more of topotecan, irinotecan, taxol, vincristin, vinblastine, etoposide, doxorubicin, danaurubicin, idarubicin, mitomycin, mitoxantrone, cisplain, cisplatin, carboplatin, procarbazine, carmustine, lomustine, DTIC, cyclophosphamide, and ifosphamide.

9. The method of any one of paragraphs 1 to 8, wherein the cyanobacteria extract is phycocyanin.

10. The method of any one of paragraphs 1 to 8, wherein the cyanobacteria extract comprises one or more of phycocyanin, allophycocyanin, and phycoerthrin.

11. The method of any one of paragraphs 1 to 10, wherein the chemotherapeutic and the cyanobacteria extract are administered simultaneously.

12. The method of paragraph 11, wherein the chemotherapeutic and the cyanobacteria extract are co-formulated.

13. The method of any one of paragraphs 1 to 10, wherein the chemotherapeutic and the cyanobacteria extract are administered sequentially.

14. The method of paragraph 13, wherein the chemotherapeutic is administered before the cyanobacteria extract.

15. The method of paragraph 13, wherein the chemotherapeutic is administered after the cyanobacteria extract.

16. The method of any one of paragraphs 1 to 15, wherein the cyanobacteria extract is administered orally, intraveneously, subcutaneously, via inhalation, or parenterally.

17. The method of any one of paragraphs 1 to 16, wherein the cancer is a carcinoma, adenocarcinoma, or sarcoma.

18. The method of any one of paragraphs 1 to 16, wherein the cancer is prostate cancer, breast cancer, ovarian cancer, colorectal cancer, lung cancer, liver cancer, pancreatic cancer, esophageal cancer, or skin cancer.

19. The method of any one of paragraphs 1 to 18, wherein the subject is a mammal.

20. The method of paragraph 19, wherein the subject is human.

21. A pharmaceutical composition comprising a cyanobacteria extract, a chemotherapeutic, and a pharmaceutically acceptable excipient.

22. The composition of paragraph 21, wherein the cyanobacteria extract comprises one or more of phycocyanin, allophycocyanin, and phycoerthrin.

23. The composition of paragraph 21 or 22, wherein the chemotherapeutic is one or more of topotecan, irinotecan, taxol, vincristin, vinblastine, etoposide, doxorubicin, danaurubicin, idarubicin, mitomycin, mitoxantrone, cisplain, cisplatin, carboplatin, procarbazine, carmustine, lomustine, DTIC, cyclophosphamide, and ifosphamide.

What is claimed is:

1. Use of a cyanobacteria extract in the preparation of a medicament for treating cancer in a subject, wherein said subject is administered a therapeutically effective amount of a chemotherapeutic and a therapeutically effective amount of the cyanobacteria extract.

2. The use of claim 1, wherein the therapeutically effective amount of the chemotherapeutic is an amount less than a therapeutically effective amount of the chemotherapeutic when administered in the absence of the cyanobacteria extract.

3. The use of claim 2, wherein the amount of the chemotherapeutic administered is 50% or less the amount administered to the subject not administered the cyanobacteria extract.

4. The use of claim 3, wherein the amount of chemotherapeutic administered is 25% or less.

5. The use of any one of claims 1 to 4, wherein the chemotherapeutic induces apoptosis.

6. The use of any one of claims 1 to 5, where the chemotherapeutic induces cytotoxicity.

7. The use of any one of claims 1 to 6, wherein the chemotherapeutic induces DNA fragmentation.

8. The use of any one of claims 1 to 4, wherein the chemotherapeutic is one or more of topotecan, irinotecan, taxol, vincristin, vinblastine, etoposide, doxorubicin, danaurubicin, idarubicin, mitomycin, mitoxantrone, cisplain, cisplatin, carboplatin, procarbazine, carmustine, lomustine, DTIC, cyclophosphamide, and ifosphamide.

9. The use of any one of claims 1 to 8, wherein the cyanobacteria extract is phycocyanin.

10. The use of any one of claims 1 to 8, wherein the cyanobacteria extract comprises one or more of phycocyanin, allophycocyanin, and phycoerthrin.

11. The use of any one of claims 1 to 10, wherein the chemotherapeutic and the cyanobacteria extract are administered simultaneously.

12. The use of claim 11, wherein the chemotherapeutic and the cyanobacteria extract are co-formulated.

13. The use of any one of claims 1 to 10, wherein the chemotherapeutic and the cyanobacteria extract are administered sequentially.

14. The use of claim 13, wherein the chemotherapeutic is administered before the cyanobacteria extract.

15. The use of claim 13, wherein the chemotherapeutic is administered after the cyanobacteria extract.

16. The use of any one of claims 1 to 15, wherein the cyanobacteria extract is administered orally, intraveneously, subcutaneously, via inhalation, or parenterally.

17. The use of any one of claims 1 to 16, wherein the cancer is a carcinoma, adenocarcinoma, or sarcoma.

18. The use of any one of claims 1 to 16, wherein the cancer is prostate cancer, breast cancer, ovarian cancer, colorectal cancer, lung cancer, liver cancer, pancreatic cancer, esophageal cancer, or skin cancer.

19. The use of any one of claims 1 to 18, wherein the subject is a mammal.

20. The use of claim 19, wherein the subject is human.

21. A pharmaceutical composition comprising a cyanobacteria extract, a chemotherapeutic, and a pharmaceutically acceptable excipient.

22. The composition of claim 21, wherein the cyanobacteria extract comprises one or more of phycocyanin, allophycocyanin, and phycoerthrin.

23. The composition of claim 21 or 22, wherein the chemotherapeutic is one or more of topotecan, irinotecan, taxol, vincristin, vinblastine, etoposide, doxorubicin, danaurubicin, idarubicin, mitomycin, mitoxantrone, cisplain, cisplatin, carboplatin, procarbazine, carmustine, lomustine, DTIC, cyclophosphamide, and ifosphamide.
